(19) United States
(12) Patent Application Publication
(10) Pub. No.: US 2023/0159966 A1
(43) Pub. Date: May 25, 2023

(54) METHOD FOR MANUFACTURING SN-2 PALMITIC TRIACYLGLYCEROLS

(71) Applicant: SOCIETE DES PRODUITS NESTLE S.A., Vevey (CH)

(72) Inventors: AMAURY PATIN, Chicago, IL (US); TIM BÖRNER, Chicago, IL (US); LARS DAHLGREN, Chicago, IL (US)

(21) Appl. No.: 17/995,318

(22) PCT Filed: Apr. 6, 2021
(86) PCT No.: PCT/EP2021/058844

§ 371 Date: Oct. 3, 2022

(30) Foreign Application Priority Data
Apr. 9, 2020 (EP) 20168959.3

(51) Int. Cl. C12P 7/6454; C12P 7/6458; C12N 9/20

(52) U.S. Cl. CPC C12P 7/6454; C12N 9/20; C12P 7/6458; C12Y 301/01003

(57) ABSTRACT

The present invention concerns an enzymatic process for the preparation of an ingredient comprising 1,3-Olein-2-palmitin (OPO), the most abundant triglyceride present in human breast milk. This is achieved by using immobilized lipase from Ther-momyces lanuginosis for producing 1,3-olein-2-palmitin (1,3-Dioleate-2-palmitate-glycerol) using as substrate tripalmitin or triglycerides enriched in palmitic acid at SN-2 position by first alcoholysis in presence of C3 to C5 alcohol (butanol, pentanol, isopropanol) to produce 2-monopalmitin which is purified by selective crystallization at decreased temperature, followed by esterification using the same lipase and oleic acid.

### FIELD OF THE INVENTION

The present invention concerns an enzymatic process for the preparation of an ingredient comprising 1,3-Olein-2-palmitin (OPO), a triglyceride present in human breast milk.

### BACKGROUND OF THE INVENTION

Triacylglycerols (TAG) are the major lipids found in human milk at about 39 g/L and they present a specific regio-specific distribution of fatty acids. The regio-specific distribution of TAG contributes to the nutritional benefits of human milk such as to fatty acid and calcium absorption and their related benefits such as gut comfort.

Infant formula (IF) ingredient design is generally aimed at structural and functional homology with respect to human milk composition and benefits.

Currently, OPO enriched ingredients are already incorporated into some IF. They are produced using enzymatic reactions (for example Betapol® or Infat®) but the OPO content in these ingredients ranges only from 20 to 28% w/w of total TAG, the rest being other TAG (for example POO, which may range from 5 to 8%w/w of total TAG). The low OPO content of these ingredients coupled with presence of other TAG represents a limit for their use in the preparation of IF having a fat portion reproducing as far as possible the fat content of human breast milk.

Other OPO synthesis are also known on lab scale using enzymatic reactions. These reactions however are either not possible to scale up at an industrial level (due to the use of large volumes of organic solvent and of complex and costly purification steps to yield the desired OPO content and/or selectivity over other TAG) or they are not capable to deliver an ingredient with desired OPO content and/or selectivity over other TAG.

There is currently no economically viable method for producing triglycerides adapted for infant formula, ideally containing more than 75% palmitic acid in the sn-2 position (also known as structured lipids). Today, such lipids for infant formula are produced via a single-step, solvent-free enzymatic acidolysis reactions where a fat high in palmitic acid is reacted with oleic acid to produce OPO. This reaction is equilibrium controlled and for high conversion yield high excess (equivalence) of oleic acid needs to be used (Akoh, 2017).

In order to modify the fatty acid composition of triacylglycerols (TAG), a lipase can be used to exchange the fatty acids in the TAG with free fatty acids added to the reaction mixture. For example, by using a sn-1(3) specific lipase on a substrate such as tripalmitin and by adding oleic acid to the reaction mixture, it is possible to produce an OPO ingredient. The main drawback with this approach is that the reaction equilibrium is thermodynamically controlled and an excess of free fatty acid is necessary to push the equilibrium towards the product side. The addition of an excess of free fatty acids drives the process cost (for example in view of additional purification steps) and/or limits the product yields possible. Betapol® and Infat® are two human milk fat mimicking commercial fats (Loders Croklaan, AAK) and are both produced by acidolysis with sn-1(3) specific lipases (Akoh, 2017).

As an alternative to produce structured lipid with high sn-2 palmitic acid content, literature describes the enzymatic two-step approach via the alcoholysis of triglycerides into 2-monoglyceride intermediate (Schmid et al, 1999) and its subsequent esterification with FFA (free fatty acids), which offers higher reaction control, purity and yield. However, this two-step process requires the use of solvents as well as costly intermediate purification steps.

Solvents are needed for two reasons: i) solubilization of the triglyceride substrate, i.e., tripalmitin and ii) for dilution to limit inhibition of the lipase by the alcohol (methanol, ethanol) in the alcoholysis step. Intermediate purification is performed either by cold fractionation in organic solvents or by distillation under strong vacuum.

In addition, the sn-2 FA content of the TAG starting material for the alcoholysis has a significant impact on the final TAG product profile. In order to maximize sn-2 palmitate in the final product, the starting material with a palmitic acid content as high as possible in position sn-2 should be used.

Thus, for an enzymatic 2-step process to become economically viable and industrially applicable, cost and starting material composition should be taken into consideration, solvent use needs to be reduced or removed and intermediate purification must be simplified, while maintaining high purity and high selectivity of the OPO ingredient obtained, for example a minimum of 50% overall OPO purity and overall a minimum of 75% of the total PA in sn-2 position.

Accordingly, there is a need to provide a novel process which would be economically viable and industrially applicable, for the preparation of an OPO ingredient with an OPO purity of at least 50 g/100 g of the ingredient and with an overall content of palmitic acid in sn-2 position which is equal or higher than 70% of total palmitic content.

### SUMMARY OF THE INVENTION

The present invention solves the above mentioned problem by providing a simplified, solvent-free, two-step enzymatic method for producing an OPO enriched ingredient with an overall content of palmitic acid in position Sn-2 larger than 70%, for example 75%. This simplified enzymatic process concept offers an economically viable route towards OPO enriched ingredient production.

In one aspect the present invention provides a process for the preparation of a 1,3-Olein-2-palmitin ingredient as described in the attached claims.

### BRIEF DESCRIPTION OF THE DRAWINGS

Additional features and advantages of the present invention are described in, and will be apparent from, the description of the presently preferred embodiments which are set out below with reference to the drawings in which:

FIG. 1 shows a schematic representation on the overall process according to one embodiment of the present invention.

FIG. 2 shows results of Example 1 and reports Yields of 2-monopalmitin over the reaction time for alcoholysis reaction using lipases Lipozyme 435 and TL IM with different alcohols. Yields calculated as mol 2-monopalmitin/mol initial tripalmitin.

FIG. 3 shows Conversion profile for isopropanolysis of tripalmitin catalyzed by Lipozyme TL IM as described in Example 1.

FIG. 4 shows each quantified species in the reaction mixture of Example 2 as a percentage of total quantified palmitic acid containing compounds.

FIG. 5 shows content of alcoholysis product compared to the precipitate from fractionation of the same mix (Example 4)

FIG. 6 shows variations of species in the reaction mixture of solvent free esterification of a 2-monopalmitic product (Example 5) based on gas chromatography data (GC).

FIG. 7 illustrates the fatty acid distribution in the final TAG mixture for Example 5 determined via LC-MS analysis.

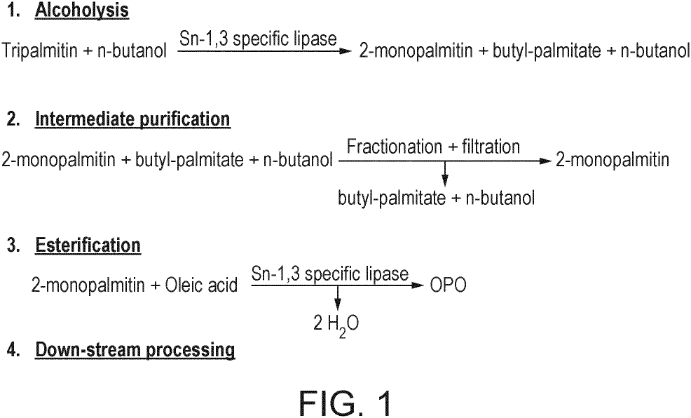

### DETAILED DESCRIPTION OF THE INVENTION

### Definitions

Within the context of the present invention the term “OPO” refers to 1,3-Olein-2-palmitin and/or 2-(palmitoyloxy)propane-1,3-diyl dioleate and/or (2-(Palmitoyloxy)-1,3-propanediyl (9Z,9′Z)bis(-9-octadecenoate) (CAS number: 1716-07-0)

Within the context of the present invention the term “POO” refers to both 3-(Palmitoyloxy)-1,2-propanediyl (9Z,9′Z)bis(-9-octadecenoate), (OOP, CAS number: 14960-35-1), and/or 1-(Palmitoyloxy)-2,3-propanediyl (9Z,9′Z)bis(-9-octadecenoate), (POO, CAS number: 14863-26-4). It is to be noted that when reference is made to amounts of “POO”, this also includes amounts of OOP present in the ingredient.

Within the context of the present invention, the term “OPO Ingredient” or “OPO enriched Ingredient” or “1,3-Olein-2-palmitin ingredient” or simply “OPO” identifies an edible ingredient comprising 1,3-Olein-2-palmitin (OPO) with purity higher than 50 g/100 g of the ingredient. In one embodiment of the present invention, the OPO ingredient prepared according to the process also has a content of palmitic acid in sn-2 position which is equal or higher than 70% of total palmitic content.

Within the context of the present invention, the term “TAG” means triacyl glycerides.

Within the context of the present invention, the term “triglycerides enriched in palmitic acid at sn-2 position” means triglycerides and/or triglyceride ingredient wherein a proportion higher than 70% of sn-2 positions in the triglycerides backbone is occupied by palmitic acid residues. In one embodiment, the triglycerides enriched in palmitic acid in sn-2 position have a proportion of sn-2 positions in the triglyceride backbone occupied by palmitic acid residues which is higher than 80%. In one embodiment, the triglycerides enriched in palmitic acid at sn-2 position is a palm oil fraction enriched in triglycerides containing palmitic acid, such as for example CristalGreen® (Bunge Loders Croklaan) which has a content of 60% w/w tripalmitin and wherein the a proportion of sn-2 positions in the triglyceride backbone occupied by palmitic acid residues which is higher than 80%.

Within the context of the present invention, the term “alcoholysis” means the transesterification reaction of fatty acids present in a triglyceride with an alcohol (methanol, ethanol, butanol...) by the action of a selective enzyme. This reaction leads to the formation of monoglycerides and fatty acid esters of the respective alcohol.

Within the context of the present invention, the term “lipase” or “sn-1,3 lipase” means a hydrolytic enzyme that acts on ester bonds (EC 3.1) and belongs to the class of carboxylic-ester hydrolases (EC 3.1.1), and more specifically possesses a high regio-selectivity for hydrolyzing the Sn-1 and Sn-3 ester bond in a triglyceride backbone. Lipases with high 1,3-selectivity can be sourced, for example, from Candidata antarctica (lipase B), Thermomyces lanuginosus, Rhizomucor miehei, R. oryza, Rhizopus delemar, etc.

Within the context of the present invention, the term “deodorization” means a steam distillation process in which steam is injected into an oil under conditions of high temperature (typically > 200° C.) and high vacuum (tipically < 20 mBar) to remove volatile components like free fatty acids (FFA), fatty acid esters, mono- and diglycerides and to obtain an odorless oil composed of TAG.

Within the context of the present invention, the term “fractionation” means a separation process in which a certain quantity of a mixture (solid, liquid, suspension) is separated into fractions during a phase transition. These fractions vary in composition thus usually allowing enrichment of a species in one of the fractions and its subsequent separation and/or purification.

Within the context of the present invention, the term “selective precipitation” or “selective crystallization” indicates a separation and/or purification technique whereby the creation of one or several specific precipitates (solids) occur from a solution containing other potential precipitates by means of adapting the temperature of the precipitation. For example, the species having a melting point above the temperature of the precipitation process will not form a precipitate under those conditions.

In one embodiment of the present invention, the selective precipitation results in crystallization of the desired product.

Within the context of the present invention, the term “immobilized form” means that the lipase enzyme is attached either covalently or non-covalently (e.g. adsorbed) to a solid carrier material. Non limiting examples of suitable carriers are:: macroporous hydrophobic supports for covalent attachment made of methacrylate resins with, for example, epoxy, butyl or amino groups together with a suitable linker molecule (e.g. glutaraldehyde); for non-covalent immobilization through hydrophobic interactions via macroporous carriers made of, e.g., polystyrenic adsorbent, octadecyl methacrylate, polypropylene, non-compressible silica gel; for non-covalently adsorption via ionic interactions ionic exchange resins are used, e.g., polystyrenic ion exchange resin or silica.

Non limiting examples of sn-1,3 lipase in immobilized form are: lipase from Thermomyces lanuginosis adsorbed on silica (e.g., Lipozyme TL IM, Novozymes), lipase B from Candida antarctica adsorbed on methacrylate/divinylbenzene copolymer (e.g. Lipozyme 435, Novozymes), lipase from Rhizomucor miehei attached via ion exchange on styrene/DVB polymer (e.g., Novozym® 40086, Novozymes) or via hydrophobic interaction onto macroporous polypropylene (Accurel EP 100).

### Alcoholysis [Step A)]

A challenge with selective alcoholysis of tripalmitin into 2-monopalmitin is the high melting point of tripalmitin (65° C.+). Chemical alcoholysis is non-specific and can thus not be used to produce 2-monopalmitin. On the contrary, enzymatic alcoholysis can lead to a highly selective alcoholysis at the sn-1,3 positions making high purity synthesis of 2-monopalmitin possible. The problem of using enzymes is the relatively poor thermostability of most of the commercial enzymes and results in lipase inactivation when reactions are performed at above 50° C. To minimize lipase inactivation and achieve full solubilization of the substrate (e.g. tripalmitin) at lower temperatures (<50° C.), organic solvents, most commonly acetone, n-hexane, or MTBE, are typically used. However, the use of solvents for industrial application increases the process complexity and operations (solvent removal and handling, safety), and thus drive the process costs (of solvent, larger reaction volumes and thus equipment/reactors) as well as pose an environmental burden (solvent recycling).

In the context of the present invention, switching from the commonly used alcohols, methanol and ethanol, to n-butanol supplied at high molar ratio (15 equivalents) has surprisingly allowed the substrate to be solubilized at 50° C. without deactivating the enzyme, producing 2-monopalmitin with 90% purity.

In one embodiment of the present invention, the alcoholysis step a) is performed with n-butanol, n-pentanol, isopropanol or mixtures thereof.

In one embodiment of the present invention, the alcoholysis step a) is performed with an excess of n-butanol.

By using n-butanol in step a) (alcoholysis), the reaction proceeded without any solvent at 50° C. Butanol acts as both substrate and solubilization agent for the triglycerides, thereby, enabling a solvent-free reaction, high conversion yield (excess) and lipase activity.

In one embodiment of the present invention, the starting material for step a) is a triglyceride mixture enriched in palmitic acid at sn-2 position, such as for example CristalGreen® (which is a commercially available product by Bunge Loders Croklaan).

In on embodiment of the present invention, step a) is performed at a temperature ranging from 40 to 70° C., for example at a temperature ranging from 45 to 55° C.

In one embodiment, step a) is performed in the presence of an sn-1,3 lipase selected in the group consisting of: lipase from Thermomyces lanuginosis adsorbed on silica (e.g., Lipozyme TL IM, Novozymes), lipase B from Candida antarctica adsorbed on methacrylate/divinylbenzene copolymer (e.g. Lipozyme 435, Novozymes) and lipase from Rhizomucor miehei attached via ion exchange on styrene/DVB polymer (e.g., Novozym® 40086, Novozymes) or via hydrophobic interaction onto macroporous polypropylene (Accurel EP 100).

In another embodiment, step a) is performed in the presence of an sn-1,3 lipase which is a lipase from Thermomyces lanuginosis adsorbed on silica (e.g., Lipozyme TL IM, Novozymes).

In step a), immobilized enzyme preparation allows to properly disperse the lipase in non-aqueous media, such as fats and solvents, and enables the recovery and reuse making the process more cost efficient.

Accordingly, alcoholysis step as described in the present invention provides several advantages to the process according to the present invention, for example:
solvent-free reaction allows smaller reactor volumes (increased volumetric productivity), lowered process costs and omits safety handling aspects, removal and recycling of the solvent (solvent removal is is especially important for an ingredient aimed at infant nutrition);
Immobilized lipases, such as Lipozyme TL IM (Novozymes), are commercially available lipases accessible at industrial scale.

### Intermediate Purification [Step B)

The two-step enzymatic transesterification process according to the present invention is more complex than conventional methods of producing OPO, e.g. single step acidolysis, yet, the moderate increase in complexity enables to improve the quality in the final product significantly, i.e. higher sn-2 palmitate content, making it more attractive for use in IF.

A two-step process requires the purification of the intermediate and it is important that the increase in quality is not offset by increase in cost potentially deriving from intermediate purification [step b)].

Current technologies for intermediate purification include molecular distillation, solvent crystallization, and chromatography but all these three methods are too costly for the targeted application and would benefit from improvement/simplification. For example, solvent fractionation methods tipically require solvent use and low temperatures (<-10° C.).

According to the process of the present invention, the intermediate purification step b) may be performed by selective crystallization of 2-monopalmitin. The side product to be removed in this purification step is product of the reaction of the alcohol (methanol, ethanol, butanol...) with the fatty acids present in position 1,3 (mainly palmitic acid). The resulting esters have different melting points depending on the alcohol used. In particular, butyl palmitate has a lower melting point (17° C.) than methyl and ethyl palmitic esters (30° C. and 24° C. respectively), providing a larger difference in melting point between 2-monopalmitin (60° C.) and the side products to be removed. This higher difference is beneficial for the separation process. Such side products including the excess of alcohol used in the alcoholysis can be effectively removed after the alcoholysis step a) by fractionation of the crude product at temperatures ranging from 0 to 10° C., whereby the 2-monopalmitin undergoes selective crystallization and the side products remain in the liquid state and can be filtered off, for example.

Accordingly, fractionation temperatures above 0° C. of the crude products and no addition of solvents allows for a simple and cheap purification step of 2-monopalmitin.

Using solvent-free fractionation, the selective crystallization of the target product (2-monopalmitin) can be performed at higher temperature and there is no need to perform a step for solvent removal by distillation.

In one embodiment of the present invention, step b) is performed by decreasing the temperature of the reaction mixture to a temperature ranging from 0 to 10° C. yielding to fractionation via selective precipitation of 2-monopalmitine and by filtering off the supernatant.

### Solvent-Free Esterification [Step C)]

Solvent free enzymatic esterification of 2-monopalmitin to form OPO has been described in literature before. In the study Highly selective synthesis of 1,3-Oleoyl-2-Palmitoylglycerol by Lipase Catalysis (Schmid et al, 1999) OPO was synthesized using sn-1,3 specific lipases from Rhizomucor miehei and Rhizopus delemar immobilized on different carrier materials. The reaction was performed at 50° C. with 3 equivalence of oleic acid and highly purified 2-monopalmitin (through solvent crystallization at -25° C.). 10-25% immobilized lipase based on weight of 2-monopalmitin was used and the authors state 78% OPO was obtained with 96% sn-2 palmitic acid using Rhizopus delemar lipase immobilized on macroporous polypropylene (EP 100) after 16 h reaction. However, the same study revealed the limited temperature stability (at 52° C.) of such immobilized R. delemar lipase and, in addition, much long reaction times were needed to reach high OPO concentrations during 2-monopalmitin esterification.

In pre-screening tests, pure 2-monopalmitin was used as starting material and three different immobilized lipases were evaluated; Lipozyme 435, Lipozyme TL IM, Novozymes 40145 NS. The most efficient lipases in forming TAGs were Novozymes NS 40 145 and TL IM.

Lipozyme TL IM was chosen for this test as it had been proven the most effective in the butanolysis reaction. With an enzyme loading of 25% w/w immobilized lipase to 2-monopalmitin, the reaction was completed after 3 h.

Additionally, asing the same lipase in both reaction steps makes the process more cost effective and allows reuse of the same immobilized enzyme preparation for both process steps a) and c). The full process from CristalGreen® to OPO could be performed using only one lipase: Lipozyme TL IM.

In step a) immobilized enzyme preparation allows to properly disperse the lipase in non-aqueous media, such as fats and solvents, and enables the recovery and reuse making the process more cost efficient.

In one embodiment of the present invention, step c) is performed at a temperature ranging from 35 to 60° C., for example at a temperature ranging from 40 to 50° C.

### Deodorization [Step D]

Deodorization of the final TAG product mixture deriving from step c) according to the process of the present invention may be performed as an optional purification step to remove the excess of free fatty acids, remaining fatty acid alkyl esters and mono- and di-glycerides.

Typically, deodorization of the mixture and/or product that needs to be purified may be performed at a temperature higher than > 200° C. and under vacuum conditions of pressure lower than 20 mBar.

### Experimental Section

### Example 1

### Production of 2-Monopalmitin via Solvent-Free Alcoholysis Under Different Conditions

### Material and Methods

Alcoholysis was performed on pure tripalmitin in solvent free conditions using isopropanol, n-butanol or and n-pentanol as alcohols.

The study was performed to assess the viability of solvent free alcoholysis of tripalmitin to produce 2-monopalmitin, using alcohols of chain length C3-C5. For the process step to be viable, high conversions must be achieved to avoid the production of side products (e.g. diglycerides) that would impact the purification process and the reaction yield.

Equipment:
10× 1.5 mL Agilent GC glass vials, screw-capped with septum
Thermomixer, with modified heating block to fit 1.5 mL Agilent GC-vials and temperature control

Chemicals:
Tripalmitin, Glycerol Tripalmitate, 98%, Alfa Aesar, LOT# 10184933
2-propanol, Honeywell, Chromasolv LC-MS
1-Butanol, Sigma-Aldrich, ≥99%
1-pentanol, Sigma-Aldrich, ≥99%

Alcohols were dried over molecular sieves (3 Å) prior to experiment.

Enzymes:
Lipozyme 435, Novozymes, Candida Antarctica lipase B immobilized on hydrophobic carrier (acryl resin)
Lipozyme TL IM, Novozymes, thermomyces lanuginosus lipase immobilized on silica gel carrier (non-compressible)

Procedure
Thermomixer was heated to 50° C.
175 mg tripalmitin was weighed into 1.5 mL glass vials with tight screw caps containing a rubber septum for sampling
Alcohol was added to the vials and closed
The closed vials were placed in the thermomixer, shaken at 650 rpm until the substrate was fully dissolved
Prior to reaction start (0 min), a sample (10 µL) was taken
Reaction was started by adding the lipase
Samples were taken after 0, 30, 60, 120, 180 and 240 minutes

Table 1 below reports lipases and alcohols used in the experiment and mass and volume in each reaction vial. Duplicate mixes were prepared, making a total of 10 vials prepared and tested.

TABLE 1

| Lipase | | | Alcohol | | |
|---|---|---|---|---|---|
| Lipase | Mass (mg) | % w/w | Alcohol | Volume (µL) | Equivalence (mol alcohol/mol tripalmtin) |
| Lipozyme 435 | 15 | 12 | 2-propanol | 300 | 18 |
| Lipozyme 435 | 15 | 12 | n-butanol | 300 | 15 |
| Lipozyme TL IM | 30 | 17 | 2-propanol | 300 | 18 |
| Lipozyme TL IM | 30 | 17 | n-butanol | 300 | 15 |
| Lipozyme TL IM | 30 | 17 | n-pentanol | 300 | 13 |

### Results and Discussion

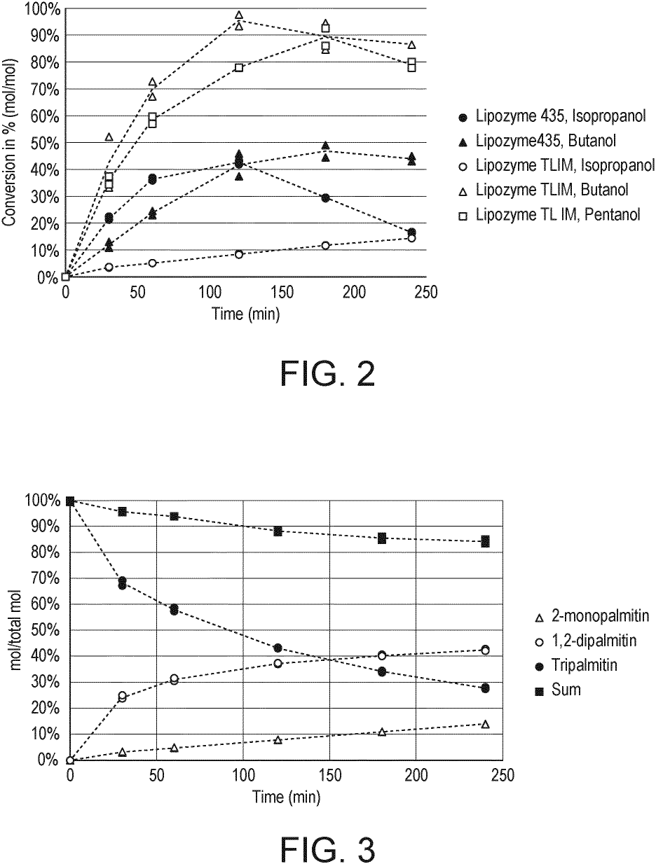

Results show that enzymatic alcoholysis of model substrate could be performed solvent free with alcohols of chain length C3-C5 using any of the lipase tested. The conversion yield of tripalmitin into 2-monopalmitin for each reaction were calculated for each sample point (and reported in FIG. 2). The best conversion yield achieved in the trial was 97%, using Lipozyme TL IM with n-butanol.

Tripalmitin was completely solubilized and miscible with the alcohols tested at 50° C. As a preliminary test, alcoholysis had been performed in ethanol, solvent-free. Because of the high melting point of tripalmitin, the reaction temperature needed to be increased to 65° C. to have a solubilized tripalmitin but under these conditions only low conversion of tripalmitin into 2-monopalmitin could be observed (33%, in the presence of Lipozyme 435, Novozymes). Attempting to dissolve tripalmitin at 50° C. by adding larger volumes of ethanol worked only poorly as the lipid and the alcohol were not fully miscible, giving a turbid suspension, and no enzymatic conversion was observed.

### Lipozyme TL IM

Higher yields were achieved using Lipozyme TL IM with the two alcohols: n-butanol and n-pentanol. The n-butanol reaction conversion reached its maximum after 2 h and the n-pentanol reaction after 3 h. The highest conversion achieved was with Lipozyme TL IM in n-butanol, reaching >95% after 2 h reaction.For Lipozyme TL IM, the reaction rates using isopropanol was lower than for the other two alcohols and the reaction didn’t run to completion.

FIG. 3 shows the amount of tripalmitin, 1,2-dipalmitin and 2-monopalmitin expressed as molar fractions of the initial glyceride content. Shown is also the sum of the three fractions.

### Lipozyme 435

The highest conversion achieved using Lipozyme 435 was below 50% after 3 h reaction with n-butanol. With isopropanol, Lipozyme 435 achieved higher reaction rates than Lipozyme TL IM. The highest conversion achieved with isopropanol was 40%, reached after 2 h reaction with Lipozyme 435.

### Example 2

### Solvent-Free Butanolysis on a Fat High in Sn-2 Palmitate by Lipozyme TL IM

Alcoholysis of a fat rich in sn-2 palmitate (CristalGreen ®) was performed to produce 2-monopalmitin in solvent-free conditions with an industrially relevant starting material.

The experiment confirmed that CristalGreen® (similarly to tripalmitin) may be a viable source of sn-2 palmitate for enzymatic production of 2-monopalmitin in reaction conditions using n-butanol and Lipozyme TL IM.

Equipment:
500 mL Schott flask with screw-cap equipped and tubing for gas sparging
Magnetic stirrer, stirrer plate
Water bath with heater/temperature control
2× 100 mL Schott flasks with rubber lined screw-caps
Adolf Kühner Lab-Therm Lab shaker with temperature control

Chemicals:
1-Butanol, Sigma-Aldrich, ≥99%, dried over molecular sieves (3 Å)
CristalGreen®

Enzymes:
Lipozyme TL IM, Novozymes, thermomyces lanuginosus lipase immobilized on silica gel carrier (non-compressible)

Procedure:

Drying CristalGreen®
100 g CristalGreen®was weighed into a 500 mL Schott flask
Flask was placed in a water bath at 70° C. and sparged with nitrogen gas for 6 h.

Reaction (duplicates)
To a 100 mL Schott flask was added:
   10 g dried CristalGreen®
   17 mL dry n-butanol
The flask was placed in a water bath at 70° C. until the fat was fully dissolved in butanol (clear, light yellow liquid)
The flask was placed in a Lab Shaker at 50° C. and 1400 rpm for 1 h
0 min sample was taken before reaction start (10 µL)
The reaction was started by adding 1.5 g Lipozyme TL IM
Samples were collected after 30, 60, 90, 120 and 150 minutes

### Lipase Reusability

High enzyme stability and reusability is one important driver for process economy and costs in enzymatic processes. Recyclability of Lipozyme TL IM was tested during alcoholysis by removing (filtration) the lipase after reaching full conversion, transferring it into a fresh substrate solution and then comparing the conversion yield and product profile for three consecutive reactions.

Procedure:

Drying CristalGreen®
100 g CristalGreen®was weighed into a 500 mL Schott flask
Flask was placed in a water bath at 70° C. and sparged with nitrogen gas for 6h.

Alcoholysis reaction was carried out in the same manner as described in example 2 i.e. 10 g dried Cristal Green was reacted with 17 mL n-butanol using 1.5 g Lipozyme TL IM as biocatalyst. The reaction was carried out for 2.5 h before being stopped. Then the reaction was stopped by filtering off the enzyme. The same enzyme was then reused in an identical reaction for three cycles. It was shown that it was possible to reuse immobilized lipase TL in three alcoholysis reactions without losing its activity as similar product profiles were obtained for each reaction cycle.

### Results and Discussion

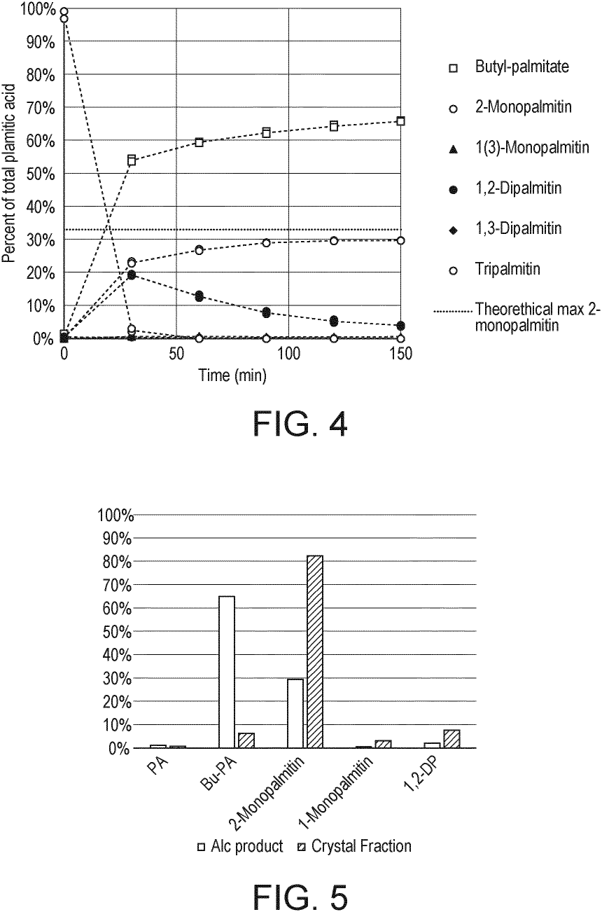

The reaction progress of the alcoholysis reaction with Cristal Green is shown in FIG. 4 and illustrates the depletion and formation of all species that contained palmitic acid (and were quantifiable by GC). The yield of 2-monopalmitin from Cristal Green in this solvent-free alcoholysis reaction amounted to 94%, based on the palmitic acid content in Sn-2 position. The starting material Cristal Green contains 32% PA in Sn-2 position (the other PA located in Sn-1 and/or 3) and 30% PA was recovered in the final 2-monopalmitin product leading to a 94% yield. The remaining 6% of PA not present in Sn-2 position was found in the few side products, i.e., 1,2-DAG and free PA quantities. The PA originally present in Sn-1 and 3 of the starting material Cristal Green were converted into palmitic acid butyl ester.

### Example 3

### Study of Purification of 2-monopalmitin by Solvent Free Fractionation (Via Selective Precipitation)

2-monopalmitin was produced by n-butanolysis of CristalGreen®using Lipozyme TL IM as described in Example 2 and purified by solvent free fractionation via selective crystallization. To the 2-monopalmitin was added 2 equivalents of fatty acid alkyl ester and 13 equivalents of alcohol to create model mixtures for the study (as described below in Table 2). These mixes were then fractionated by gradually lowering the temperature in a water bath.

TABLE 2

| Palmitic acid Alkyl Ester | Weighed: (mg) | n: (mmol) | n(2-MAG) (mol) | (2-MAG) to weigh (mg): | n(alcohol) (mol) | m(alcohol) (mg) | V(alcohol) (mL) | V(alcohol) (µL) |
|---|---|---|---|---|---|---|---|---|
| Methyl- | 1058 | 3.91 | 1.96 | 645 | 0.0254 | 815 | 1.029 | 1029 |
| Ethyl- | 644 | 2.27 | 1.13 | 374 | 0.0147 | 679 | 0.860 | 860 |
| isopropyl- | 722 | 2.42 | 1.21 | 399 | 0.0157 | 946 | 1.203 | 1203 |
| n-butyl- | 600 | 1.92 | 0.96 | 317 | 0.0125 | 926 | 1.1444 | 1144 |
| n-pentyl- | 796 | 2.44 | 1.22 | 402 | 0.0159 | 1398 | 1.723 | 1723 |

### Part I - Preparing Palmitic Acid Alkyl Esters

Fatty acid alkyl esters were prepared from palmitic acid and alcohols methanol, ethanol, isopropanol, n-butanol and n-pentanol. The reaction was run in MTBE for the methanol and ethanol reactions. The other reactions were run solvent free. Lipozyme 435 catalyzed the reactions.

Equipment
5x 100 mL Schott flask with rubber lined screw-caps
Adolf Kühner Lab-Therm Lab shaker with temperature control
Büchi rotavapor - lab scale evaporator
Vacuum filtration setup
5× 50 mL round flasks

One gram of palmitic acid was reacted using 1 gram of Lipozyme 435 in 10 mL alcohol for isopropanol, butanol, and pentanol. Methanol and ethanol preparations were performed with 1 mL alcohol and 10 ml MTBE. Molecular sieves (3 Å) were added to the mixtures for water removal.

The reaction was performed at 50° C. with a shaking of 1400 rpm. The reaction was started by adding the lipase and ran for 12 hours. The reaction was stopped by filtering off the lipase. After the reaction was stopped, the remaining alcohols and solvents were evaporated in a rotavapor.

The retained phase from the evaporation was transferred to clear 2 mL glass vials and weighed. The corresponding amounts of 2-monopalmitin and alcohol were calculated and added to the tubes as per Table 2.

### Part II - Crystallization/Fractionation Behavior of Mixtures of Fatty Acid Alkyl Ester, 2-Monopalmitin And Various Alcohols

The mixes prepared under Part I were placed in a water bath at 40° C. The temperature was then gradually lowered and the phase transitions of the mixes and precipitation behavior were observed for the following 5 mixtures (as per Table 2):
methyl palmitate + 2-monopalmitate + methanol
ethyl palmitate + 2-monopalmitate + ethanol
isopropyl palmitate + 2-monopalmitate + isopropanol
n-butyl palmitate + 2-monopalmitin + n-butanol
n-pentyl palmitate + 2-monopalmitin + n-pentanol

Melting points:
2-monopalmitin: 65° C.
Methyl palmitate: 30° C.
Ethyl palmitate: 24° C.
n-propyl palmitate: 20.4° C.
n-butyl palmitate: 16.9° C.

### Results and Discussion

As a result of the experiment, isopropyl-, n-butyl- and n-pentyl- mixtures could be fractionated, as 2-monopalmitin and 1,2-dipalmitin precipitated while the alcohol and its corresponding palmitic acid alkyl ester remained in solution. Methyl- and ethyl- mixes could not be fractionated.

The mixtures deriving from longer chain alcohols formed white crystals of 1,2-dipalmitin and 2-monopalmitin.

The mixtures deriving from shorter chain could not be fractionated but rather the whole mix solidified.

From these results, it can be inferred that using a longer chain alcohol in the alcoholysis step aids in fractionation and makes solvent free fractionation possible.

Accordingly, having a C3-C5 alcohol gives the additional unexpected benefit of an simplified intermediate purification step for the desired product (2-monopalmitin).

### Example 4

### Intermediate Purification - Solvent-Free Fractionation via Selective Crystallization of Product Mixture Obtained By Solvent-Free Butanolysis of CristalGreen®

This study was performed to purify 2-monopalmitin from the product of the alcoholysis step as described in Example 2 via solvent free fractionation via selective precipitation.

Equipment:
50 mL Erlenmeyer flask
Vacuum filtration setup with xxx glass filter

Chemicals:
From the alcoholysis step as described in Example 2, a final reaction mixture is obtain after 2.5 h reaction consisting of approximately 0.95 equivalences 2-monoglycerides, 0.05 eq. 1,2-diglycerides, 2 eq. fatty acid n-butyl esters, 13 eq. n-butanol
n-heptane

Procedure:
The alcoholysis reaction was stopped by filtering of the lipase
The filtrate was transferred to a 50 mL Erlenmeyer flask
The flask was placed at 4° C. overnight
Part of the fractionation mix was poured over the glass filter. The solution passes through, leaving a filter cake of white crystals. The crystals were washed by dripping heptane over them while still running the vacuum. The vacuum was then stopped and the crystals scraped off the filter.
The crystals were dried in a desiccator and weighed.

Recovered from the fractionation and filtration was 1.62 g crystal fraction.

The achieved overall process yield as described in Examples 2 and 4 was 40%.

### Result and Discussion

Intermediate purification by fractionation via selective crystallization of 2-monopalmitin was successfully performed on the final reaction mix from butanolysis of CristalGreen®. The amount of butyl palmitate was reduced by 90%. This shows that the method is viable for separating 2-monopalmitin (crystals) from liquid butyl palmitate and butanol, for example, via filtration.

### Example 5

### Solvent-Free Esterification With Oleic Acid of 2-Monopalmitin Product Derived from Butanolysis for OPO Ingredient Production

The present experiment was performed to demonstrate that 2-monopalmitin produced by butanolysis from CristalGreen® (as described in Example 2), purified by solvent-free fractionation via selective crystallization (as described in Example 4), can be successfully enzymatically esterified with oleic acid to produce OPO. The final ingredient contains a Sn-2 palmitate content matching that of human breast milk (70% or higher).

Equipment:
2× 25 mm Pyrex glass tubes with rubber caps equipped with tubing for gas sparging
Water bath with heater/temperature control

Chemicals:
Oleic acid, ≥99%, Sigma-Aldrich, LOT# 0000051240
2-monopalmitin, produced through butanolysis from CristalGreen®, purified by solvent free fractionation via selective crystallization

Enzymes:
Lipozyme TL IM, Novozymes, Thermomyces lanuginosus lipase immobilized on silica gel carrier (non-compressible)

Experiment:
Water bath was heated to 45° C.
Added to Pyrex 25 mL glass tube:
   1 g 2-monopalmitin
   2.5 mL oleic acid (approx. 2.6 eq.)
Pyrex tubes were placed in the water bath with nitrogen gas sparing through the oil 2-monopalmitin/oleic acid mix until the mix turned clear, the 2-monopalmitin was fully solved
The reaction was started by addition of 250 mg Lipozyme TL IM (25% w/w)

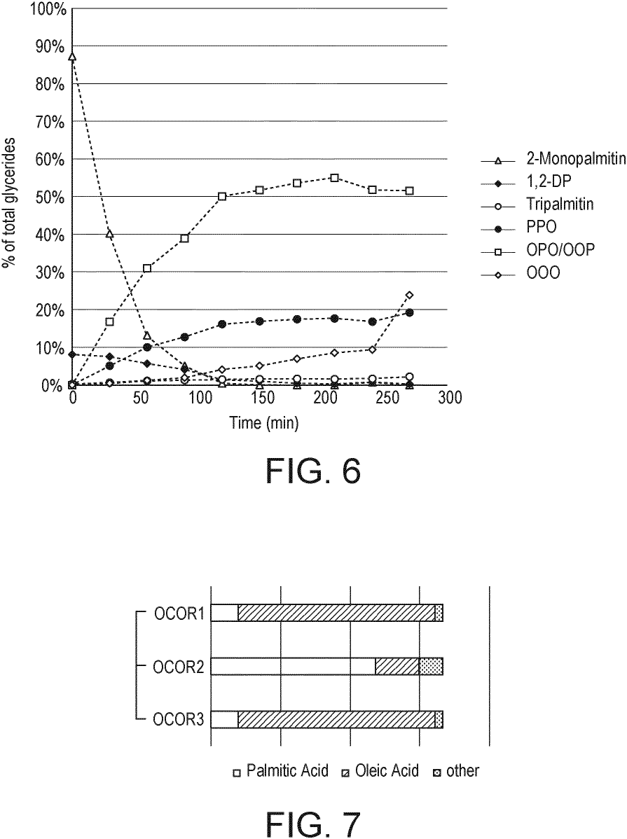

FIG. 6 shows the conversion profile of the reaction based on gas chromatography (GC) analysis; the amounts of each glyceride is presented as percent of total glycerides. FIG. 6 shows 2-monopalmitin decreasing and being fully depleted after 2 h reaction.

As GC analysis method cannot distinguish between OPO and POO, further analysis using LC-MS was carried out on the final mixture showing it mostly contained OPO. The fatty acid distribution in the final TAG mixture is illustrated in FIG. 7.

### Results and Discussion

2-monopalmitin produced by butanolysis of CristalGreen® and purified by fractionation (as describe din Example 2 and 4) was enzymatically esterified with oleic acid to form OPO.

The final triglyceride profile of the obtained product consisted of 60% OPO and 75% sn-2 palmitic acid.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

1. A process for the preparation of a 1,3-Olein-2-palmitin ingredient comprising the steps of:
a) subjecting tripalmitin and/or triglycerides enriched in palmitic acid at sn-2 position to an alcoholysis step performed in the presence of an immobilized lipase and of a primary or secondary alcohol of chain length C3-C5, to give a product mixture comprising 2-monopalmitin;
b) purifying the mixture comprising 2-monopalmitin obtained in step a) by fractionation process via selective crystallization of 2-monopalmitin and subsequent removal of the remaining liquid fraction (supernatant); and
c) subjecting the mixture deriving from step b) to an esterification step in the presence of oleic acid and of an immobilized lipase to produce the 1,3-Olein-2-palmitin ingredient.

2. A process according to claim 1 wherein alcoholysis of step a) is performed with n-butanol in the presence of Thermomyces lanuginosis adsorbed on silica.

3. A process according to claim 1 wherein the starting material for step a) is triglycerides enriched in palmitic acid at sn-2 position .

4. A process according to claim 1 wherein step a) is performed at a temperature ranging from 40 to 70° C.

5. A process according to claim 1 wherein step b) performed by decreasing the temperature of the mixture to a temperature ranging from 0 to 15° C., to allow fractionation via selective crystallization of 2-monopalmitin and by removing the remaining liquid fraction.

6. A process according to claim 1 wherein step c) is performed at a temperature ranging from 35 to 60° C., in the presence of Thermomyces lanuginosis adsorbed on silica.

7. A process according to claim 1 wherein step c) is followed by a deodorization step d).
